(19) United States
(12) Patent Application Publication
(10) Pub. No.: US 2026/0241163 A1
(43) Pub. Date: Aug. 20, 2026

(54) PACING WIRE CLIP WITH INTEGRATED INSULATION

(71) Applicant: BOSTON SCIENTIFIC SCIMED, INC., Maple Grove, MN (US)

(72) Inventors: Andrew John Mitterholzer, Eden Prairie, MN (US); Luis Gabriel Lopez-de-Victoria, Maple Grove, MN (US)

(73) Assignee: BOSTON SCIENTIFIC SCIMED, INC., MAPLE GROVE, MN (US)

(21) Appl. No.: 19/544,294

(22) Filed: Feb. 19, 2026

Related U.S. Application Data
(60) Provisional application No. 63/760,390, filed on Feb. 19, 2025.

(51) Int. Cl. A61N 1/05

(52) U.S. Cl. CPC A61N 1/0565

(57) ABSTRACT

A pacing clip device for use with a guidewire during transcatheter procedures includes a clip body having first and second arms pivotally connected to transition between open and closed positions. The device includes a conductive connector housed within at least one arm, featuring at least one end with a semicircular cutout configured to engage a guidewire core wire. A spring mechanism holds the arms in the closed position. The device further includes an insulating tube that extends proximally from the clip body. The insulating tube provides electrical isolation and coverage of exposed portions of the guidewire when extended, while the conductive connector establishes secure electrical contact with the guidewire. This configuration enables reliable electrical connectivity for pacing procedures.

### CROSS REFERENCE TO RELATED APPLICATIONS

This application claims the benefit of priority of U.S. Provisional Application No. 63/760,390 filed February 19, 2025, the entire disclosure of which is hereby incorporated by reference.

### TECHNICAL FIELD

The present disclosure generally relates to establishing electrical connections during transcatheter procedures. Particularly, but not exclusively, the present disclosure relates to strengthening electrical connections of a pacing system used during transcatheter procedures via a simplified circuit configuration.

### BACKGROUND

Transcatheter procedures often require establishing temporary electrical connections to provide cardiac pacing. Traditional approaches may utilize electrical connectors to establish the necessary electrical connections between components of a pacing system.

Current methods of establishing electrical connections during procedures like transcatheter aortic valve replacement (TAVR) can result in inconsistent electrical contact and potential signal loss, increasing procedural complexity and patient discomfort. There remains a need for reliable solutions that can maintain consistent electrical contact between the components of a pacing system.

### BRIEF SUMMARY

This overview is intended to provide an introduction to the subject matter of the present patent application. It is not intended to provide an exclusive or exhaustive explanation. The detailed description is included to provide further information about the present patent application.

In an example, a pacing clip device for use with a guidewire during transcatheter procedures includes a clip body having a first arm pivotally connected to a second arm to transition between an open position and a closed position, a conductive connector housed within at least one of the first and second arms and having at least one semicircular cutout configured to electrically engage the guidewire, a spring mechanism configured to hold the first and second arms in the closed position, and an insulating tube extending proximally from the clip body.

Alternatively or additionally to any of the examples above, the semicircular cutout is configured to establish electrical contact with a core wire of the guidewire between insulating wrappings of the guidewire when the clip body is in the closed position.

Alternatively or additionally to any of the examples above, the conductive connector includes a distal prong and a proximal prong individually configured to engage a space between turns of the insulating wrappings on the guidewire and mechanically and electrically contact the core wire.

Alternatively or additionally to any of the examples above, the first arm includes a first top portion and a first bottom portion, and the second arm includes a second top portion and a second bottom portion, wherein the first top portion and the second top portion are moved toward a central axis of the guidewire and the first bottom portion and the second bottom portion are moved apart relative to the central axis of the guidewire when the clip body is in the open position, and wherein the first top portion and the second top portion are moved apart relative to the central axis of the guidewire and the first bottom portion and the second bottom portion are moved toward the central axis of the guidewire when the clip body is in the closed position.

Alternatively or additionally to any of the examples above, the insulating tube includes an accordion-like structure with alternating ridges and valleys configured to transition from a compressed configuration to an extended configuration.

Alternatively or additionally to any of the examples above, the insulating tube is in the compressed configuration when the pacing clip device is loaded onto the guidewire.

Alternatively or additionally to any of the examples above, the insulating tube is configured to cover at least an exposed portion of a core wire of the guidewire when the insulating tube is in the extended configuration.

Alternatively or additionally to any of the examples above, the insulating tube includes a proximal portion having an accordion-like structure with alternating ridges and valleys configured to extend proximally along the guidewire, and a distal portion lacking the accordion-like structure configured to be coupled with the clip body.

Alternatively or additionally to any of the examples above, the insulating tube includes a longitudinal split extending along its length to allow for lateral placement of the insulating tube onto the guidewire when the clip body is in the open position.

In an example, a method of attaching a pacing clip device to a pacing system during a transcatheter procedure includes attaching a clip body to a guidewire, the clip body including a first pivotable arm and a second pivotable arm, a conductive connector disposed within at least one of the first and second pivotable arms, the conductive connector having at least one end surface defining a cutout, transitioning the clip body to an open position, positioning the clip body onto the guidewire while in the open position, transitioning the first and second pivotable arms to a closed position by pivoting the first pivotable arm and the second pivotable arm relative to each other, wherein at least one cutout is engaged with a core wire of the guidewire when the clip body is in the closed position, and extending an insulating tube proximally from the clip body along the guidewire from a compressed configuration to an extended configuration over an exposed proximal portion of the guidewire.

Alternatively or additionally to any of the examples above, the conductive connector includes a distal prong and a proximal prong each defining the at least one end surface defining the cutout, wherein the distal and proximal prongs are configured to be inserted between insulating wrappings of the guidewire when the clip body is in the closed position.

Alternatively or additionally to any of the examples above, the distal prong and the proximal prong are in contact with the core wire of the guidewire when the clip body is in the closed position.

Alternatively or additionally to any of the examples above, extending the insulating tube includes transitioning the insulating tube from the compressed configuration to the extended configuration to cover at least an exposed portion of the core wire of the guidewire.

Alternatively or additionally to any of the examples above, positioning the clip body further includes laterally placing the clip body onto the guidewire and laterally placing the insulating tube onto the guidewire through a longitudinal split in the insulating tube.

In an example, a system to establish an electrical connection in a pacing system includes a guidewire having a guidewire coil wrapped around a core wire and a pacing clip device including a clip body having a first arm and a second arm pivotally connected to transition between an open position and a closed position, a conductive connector housed within at least one of the first and second arms and having at least one end with a semicircular cutout configured to engage the core wire between turns of the guidewire coil, a spring mechanism configured to hold the first and second arms in the closed position, and an insulating tube extending proximally from the clip body.

Alternatively or additionally to any of the examples above, the semicircular cutouts are configured to mechanically and electrically engage with the core wire between wrappings of the guidewire coil of the guidewire when the clip body is in the closed position.

Alternatively or additionally to any of the examples above, the insulating tube includes a longitudinal split configured to enable lateral placement of the insulating tube onto the guidewire.

Alternatively or additionally to any of the examples above, the insulating tube includes an accordion-like structure with alternating ridges and valleys configured to extend proximally along the guidewire after transitioning the insulating tube from a compressed configuration to an extended configuration.

Alternatively or additionally to any of the examples above, the insulating tube is configured to cover an exposed portion of the core wire of the guidewire when the insulating tube is in the extended configuration.

Alternatively or additionally to any of the examples above, the first and second arms are configured to transition between the open position for lateral placement of the guidewire and the closed position wherein the conductive connector engages the guidewire to establish electrical contact.

### BRIEF DESCRIPTION OF THE SEVERAL VIEWS OF THE DRAWINGS

FIG. 1A is an end view of a clip body in an open position in accordance with at least one embodiment of the subject matter disclosed herein.

FIG. 1B is an end view of the clip body of FIG. 1A in a closed position on a guidewire in accordance with at least one embodiment of the subject matter disclosed herein.

FIG. 2A is a side view of a pacing clip device on a guidewire in accordance with at least one embodiment of the subject matter disclosed herein.

FIG. 2B is an end view of the pacing clip device of FIG. 2A.

FIG. 3A is a side view of a pacing clip device with an insulating tube in a compressed configuration in accordance with at least one embodiment of the subject matter disclosed herein.

FIG. 3B is a bottom view of the insulating tube of FIG. 3A.

FIG. 3C is a side view of the insulating tube in an expanded configuration in accordance with at least one embodiment of the subject matter disclosed herein.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

A conventional pacing system consists of an external pulse generator (herein referred to as “EPG”), a positive lead connected to a guidewire, and a negative lead connected to a patient. Electrical stimulus begins at the positive terminal of the EPG and flows through the positive lead and the guidewire to a target treatment location. In a transcatheter aortic valve replacement ("TAVR") procedure, the target treatment location is heart vasculature, which may include the aortic valve annulus, the aortic root, ascending aorta, and the aortic arch. The access site for advancement of the delivery catheter may include, but is not limited to, a femoral artery access site, subclavian artery access site, carotid artery access site, apical access site, transaortic access site, transhepatic access site, and direct coronary sinus access site.

In conventional pacing systems, the positive lead and the guidewire are connected by an electrical connector. Examples of electrical connectors include alligator clips, snap connectors, banana plugs, screw-type connectors, pin and socket connectors, spring loaded clamps, or other connectors made of conductive material. However the use of an electric connector in a traditional manner may present issues. For example, a second user may be required to connect and disconnect the positive pacing lead from the guidewire due to the distance between a patient vasculature access site and the proximal end of the guidewire. Depending on the type of electric connector, the electrical connector may also be attached in mechanically insecure manner, which may potentially lead to loss of signal during the procedure.

By adding a pacing clip device that includes a conductive connector to directly engage with the guidewire, the pacing clip device may serve the role of the electrical connector of the conventional pacing system and provide a more secure connection without the use of a secondary user may be accomplished.

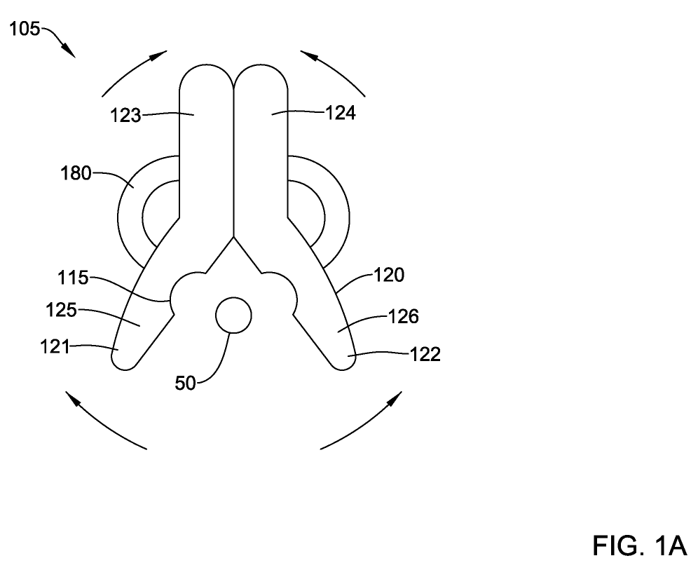

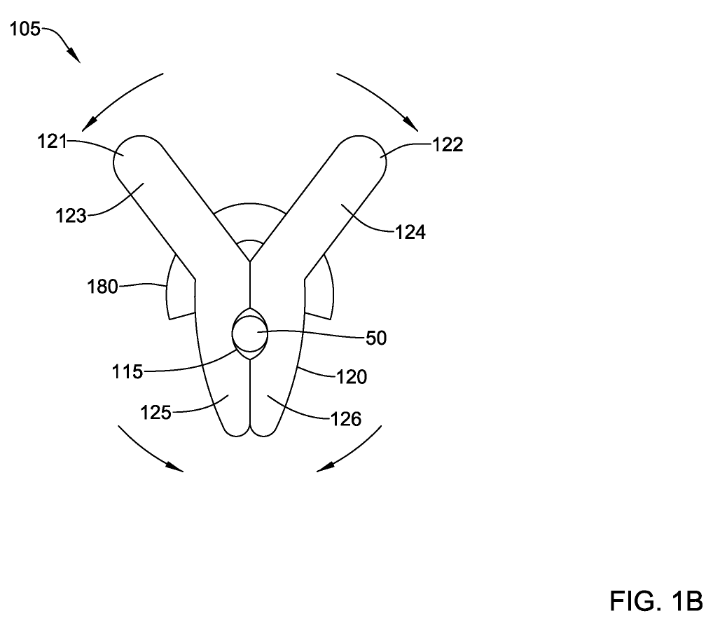

FIG. 1A and FIG. 1B show an end view of the pacing clip device 105 in an open position and closed position, respectively.

A pacing clip device 105 includes a clip body 120 including a first arm 121 and second arm 122 which are connected by a spring mechanism 180. Prior to operation, the clip body 120 is biased in the closed position, as illustrated and discussed later in FIG. 1B.

As illustrated in FIG. 1A, an user may prepare to load the pacing clip device 105 to a guidewire 50 by transitioning the clip body 120 to the open position. The first arm 121 and the second arm 122 are pivotally connected such that the user may pivot the first and second arms 121, 122 to move them relatively towards each other along the central axis of the guidewire 50 (e.g., the user “pinches” the first and second arms 121, 122 closer together). When the user pinches a first top portion 123 of the first arm 121 and the second top portion 124 of the second arm 122, together, a first bottom portion 125 of the first arm 121 and a second bottom portion 126 of the second arm 122 move away from each other and away from the central axis of the guidewire 50, thereby releasing the guidewire 50. At least one of the first and second arms 121, 122 may include a conductive connector 115 configured to provide an electrical connection with the guidewire 50 when the clip body 120 is closed on the guidewire 50.

The first and second arms 121, 122 of the clip body 120 may be made of a suitable non-conductive material of sufficient rigidity to allow for transition of the clip body 120 to the open position without deformation of either of the first and second arms 121, 122. Examples of appropriate materials include, but are not limited to, polycarbonate, nylon/polyamides, PTFE, polypropylene, polyvinyl chloride (PVC), acrylic, polyethylene terephthalate (PET), polyphenylene oxide, polybutylene terephthalate (PBT), polyamide-imide (PAI), polyetheretherketone (PEEK).

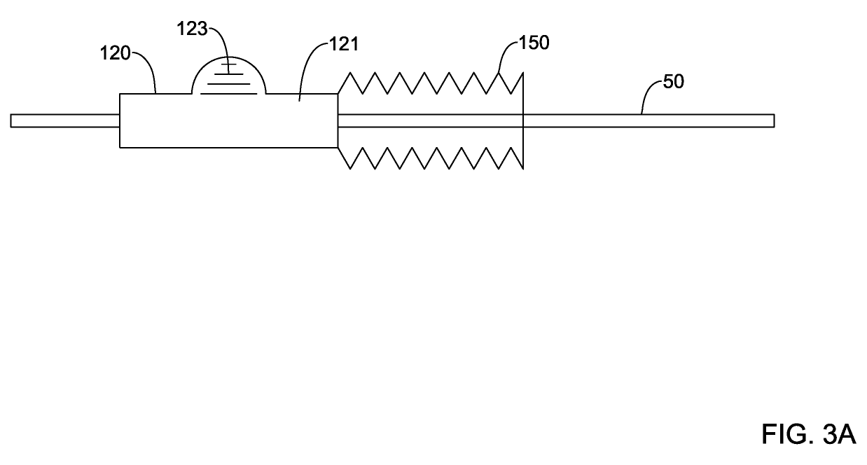

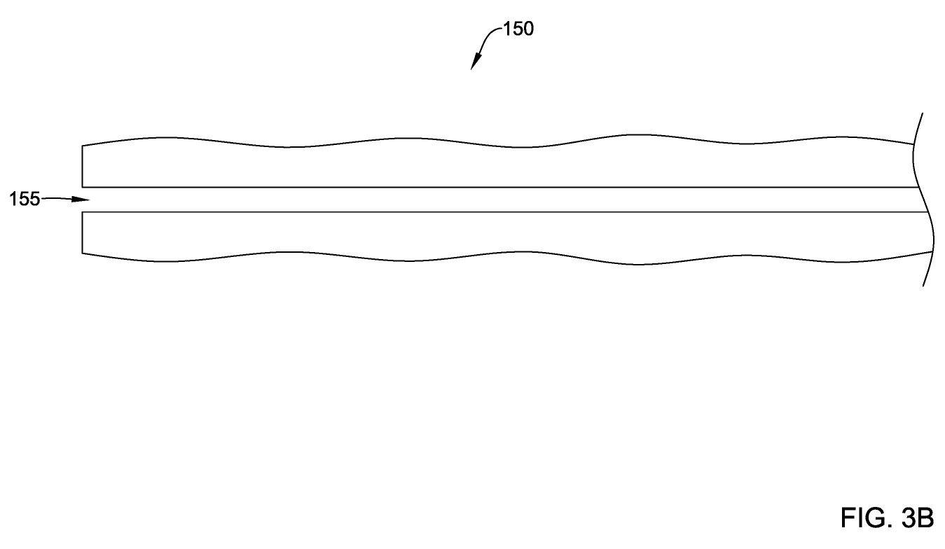

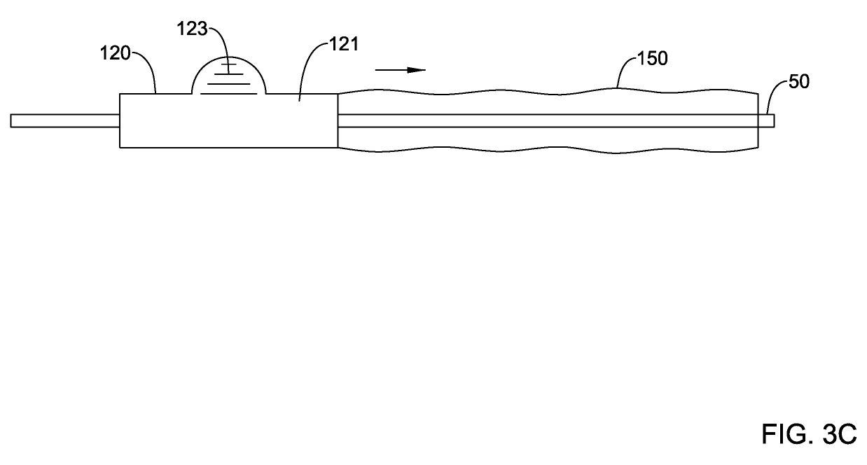

The user may prepare to load the pacing clip device 105 to the guidewire 50 by transitioning the clip body 120 to the open position. The user may spread the first and second arms 121, 122 apart by consequently moving the first top portion 123 of the first arm 121 and the second top portion 124 of the second arm 122 towards each other. To facilitate this transition, the top portions 123,124 may include a textured surface to improve grip and control over this motion (see side view of the first arm 121 and the first top portion 123 illustrated in FIG. 3A). Textured surfaces include, but are not limited to, ribbed, corrugated, or grooved (including helical, chevron, or knurled grooves).

Simultaneously with the movement of the top portions 123, 124, the first bottom portion 125 of the first arm 121 and the second bottom portion 126 of the second arm 122 move apart relative to each other and away from the central axis of the guidewire 50, completing the transition to the open position, shown in FIG. 1A.

The user may load the pacing clip device 105 to the guidewire 50 by transitioning the clip body 120 to the closed position around the guidewire 50, as illustrated in FIG. 1B. The closed position also resembles the arrangement of the clip body 120 in the closed position prior to the transition of the clip body to the open position for loading.

In the closed position shown in FIG. 1B, the top portions 123, 124 are apart relative to each other. To transition to the closed position, the user releases the top portion 123 of the first arm 121 and the top portion 124 of the second arm 122, and the spring mechanism 180 pushes the top portion 123 of the first arm 121 and the top portion 124 of the second arm 122 relatively away from each other. Simultaneous with the movement of the top portions 123, 124, the first bottom portion 125 of the first arm 121 and the second bottom portion 126 of the second arm 122 move toward each other and towards the central axis of the guidewire 50 to grip the guidewire 50.

These transitional movements described in FIG. 1A and FIG. 1B are facilitated by the spring mechanism 180. When the top portions 123, 124 of the first and second arms 121, 122 are pinched together in the open position, the spring mechanism 180 is compressed. Once the pinching force is released, pressure on the spring mechanism 180 is relieved, and the spring mechanism 180 pushes the top portions 123, 124 apart relative to each other, consequently moving the first bottom portion 125 of the first arm 121 and the second bottom portion 126 of the second arm 122 relatively toward each other to engage the guidewire 50. In some embodiments, the spring mechanism 180 may be a torsion spring mechanism, a compression spring mechanism, or a double-spring mechanism. The spring mechanism may be made of a non-conductive or conductive material suitable to withstand the pinching forces placed on the spring mechanism 180. The spring mechanism 180 biases the clip body 120 in its closed position.

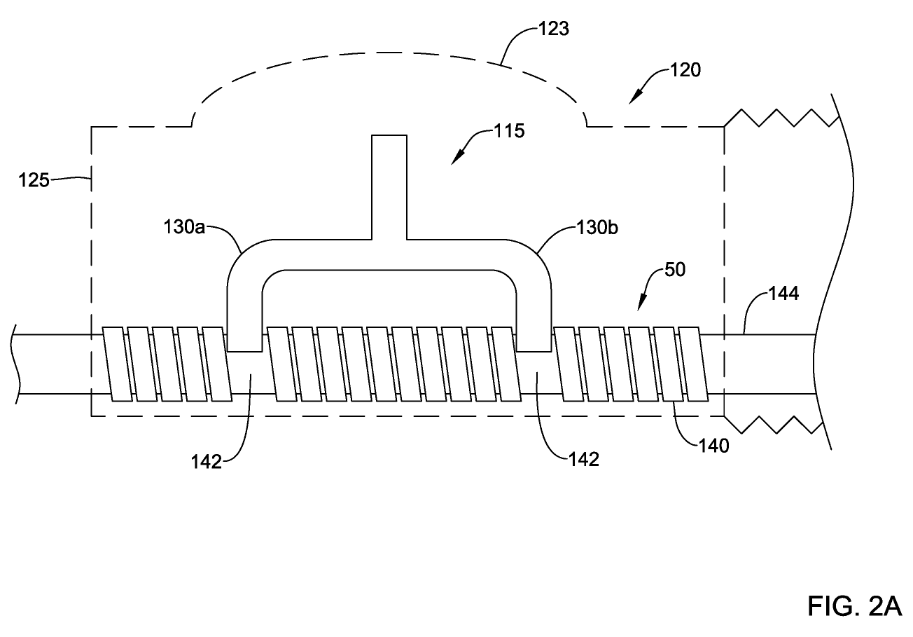

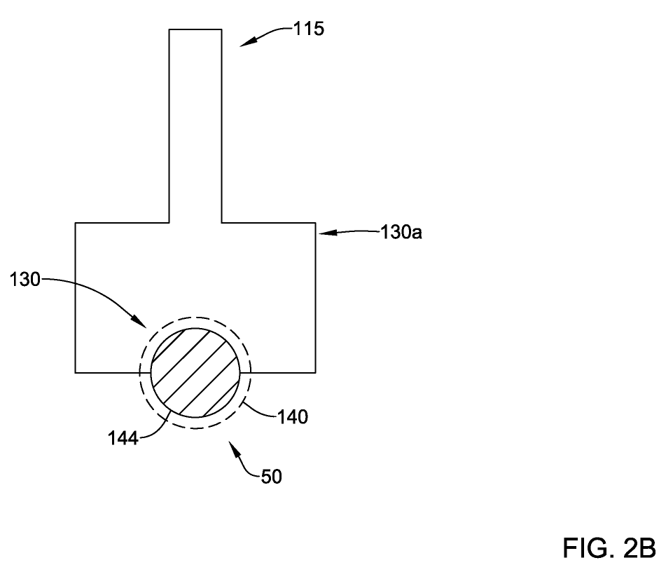

At least one of the first and second arms 121, 122 may include a conductive connector 115 housed within the arm to axially engage the guidewire 50 when the clip body 120 is in the closed position. In FIGS. 1A, 1B and 2A, the conductive connector 115 is disposed within the first arm 121, however it will be understood that the conductive connector 115 may be provided within the second arm 122, or conductive connectors 115 may be provided within both the first and second arms 121, 122.

The engagement of the conductive connector 115 with the guidewire 50 is illustrated in FIG. 2A, with the clip body 120 including the top portion 123 and bottom portion 125 of the first arm shown in phantom lines in order to visualize the inner components.

Guidewire 50 includes a core wire 144 and a guidewire coil 140. The core wire 144 may extend from a proximal end of the guidewire 50 to the distal end of the guidewire 50. The core wire 144 may be structured as a solid wire, typically cylindrical in shape. The core wire 144 may be manufactured with an appropriate material to provide the necessary electrical conductivity for operation of the pacing system. The core wire 144 may be manufactured from, for example, but not exclusively, of appropriate conductive material including, but not limited to, stainless steel, titanium, tantalum, tungsten, gold, platinum, palladium, and combinations thereof; and the metal alloy is selected from the group consisting of nickel-titanium alloys, nickel-chromium alloys, nickel-chromium-iron alloys, cobalt-chromium-nickel alloys, nickel-chromium-molybdenum alloys, nickel-molybdenum alloys, nickel-copper alloys, cobalt-chromium alloys, cobalt-chromium-molybdenum alloys, platinum-enriched stainless steel, and combinations and alloys thereof. Other considerations in the selection of the material may include, but are not limited to, strength, flexibility, and corrosion resistance.

The guidewire coil 140 spirals in a helical, co-centric fashion around the core wire 144, and may be made of a non-conductive or insulating material wrapped around the core wire 144. The guidewire coil 140 may be secured to the core wire 144 at each of its ends using a bonding agent or remain free-floating relative to the core wire 144. The guidewire coil 140 may extend for the length of the guidewire 50 or a partial length of the guidewire 50.

The guidewire coil 140 includes turns of the guidewire coil 140. The guidewire 50 is sufficiently compressible such that the turns of the guidewire coil 140 may be compressed to create spaces 142 between them, allowing for mechanical and electrical engagement with the core wire 144 through these spaces 142.

In some embodiments, the conductive connector 115 may include a distal prong 130a and a proximal prong 130b, each having an end surface defining a cutout 130. When the clip body 120 is in the closed position, the distal prong 130a and the proximal prong 130b may be configured such that the cutout 130 (FIG. 2B) on the end of each prong engages the core wire 144 between turns of the guidewire coil 140 such that distal prong 130a and proximal prong 130b are each in contact with the core wire 144, thus providing a secure electrical continuity between the pacing clip device 105, the guidewire 50, and the positive lead once the pacing clip device 105 is in the closed position.

The distal prong 130a and proximal prong 130b are sized to be able to fit in between the turns of guidewire coil 140. In some embodiments, the guidewire coil 140 is sufficiently axially compressible to create spaces 142 between the turns of the guidewire coil 140 at the location where the prongs 130a, 130b are engaged with the core wire 144, while the remaining turns of the guidewire coil 140 are compressed once the clip body 120 has been transitioned into its closed position. The distal prong 130a and the proximal prong 130b may also aid in locating the turns of the guidewire coil 140 and aid in creating larger spaces 142 between the turns of the guidewire coil 130 to further ensure mechanical and electrical contact and engagement with the core wire 144.

The conductive connector 115 may be manufactured from, for example, but not exclusively, of appropriate conductive material including, but not limited to, stainless steel, titanium, tantalum, tungsten, gold, platinum, palladium, and combinations thereof; and the metal alloy is selected from the group consisting of nickel-titanium alloys, nickel-chromium alloys, nickel-chromium-iron alloys, cobalt-chromium-nickel alloys, nickel-chromium-molybdenum alloys, nickel-molybdenum alloys, nickel-copper alloys, cobalt-chromium alloys, cobalt-chromium-molybdenum alloys, platinum-enriched stainless steel, and combinations and alloys thereof.

FIG. 2B is an end view of the conductive connector 115 engaged with the guidewire 50, shown without the other components of the pacing clip device for clarity. The end view of FIG. 2B shows the distal prong 130a in the forefront. However, it should be understood that this view is not affected when observed from the opposite end, where the proximal prong 130b is in the forefront.

The semicircular shape of the cutout 130 may be designed to conform to the cylindrical shape of the core wire 144, enabling direct metal-to-metal contact between the prong 130a and the core wire 144 when engaged between the guidewire coil 140 turns, which are shown in phantom lines to allow viewing the core wire 144. This configuration provides secure electrical continuity between the pacing clip device and the guidewire 50 when the clip body is in the closed position.

In a TAVR procedure, the guidewire is typically insulated in order to ensure electrical stimuli is delivered specifically to the target treatment location, maintain the EPG as the source of pacing (rather than the heart), reduce electrical hazards, interference with electrical equipment, damage to vasculature structures, and improve control and steerability. To retain the benefits of an insulated guidewire when using the disclosed pacing system, an insulating tube 150 may be used in conjunction with the clip body 120, as illustrated in FIG. 3A and FIG. 3B, to cover the guidewire 50 extending proximal of the clip body 120. In some embodiments (and as illustrated), clip body 120 may be placed on the guidewire 50, adjacent the vasculature access site. Once the clip body 120 is placed on the guidewire 50, the insulting tube 150 may be placed proximal of the clip body 120 and extended proximally to cover a portion of the guidewire 50. In this example, the clip body 120 and the insulating tube 150 are ultimately placed adjacent the vasculature access site and distal of other procedural devices when the insulating tube 150 is extended proximally. In other embodiments, the clip body 120 may be placed at the proximal end of the guidewire 50 (proximal of the patient vasculature access site). In this example, the insulating tube 150 may be placed distal of the clip body 120 and extended distally up to, but not through, the vasculature access site.

The insulating tube 150 is an accordion-like structure with alternating ridges and valleys. The structure of insulating tube 150 may allow for proximal or distal compression and extension of the insulating tube 150. The insulating tube 150 may be made of a thin polymer material that is sufficiently flexible to allow the ridges of the insulating tube 150 to be compressed and extended. The insulating tube 150 may be coupled to the proximal end or distal end of the clip body 120. In some embodiments, the insulating tube 150 may be fixedly attached to the clip body 120 such as with adhesive or heat bonding. In other embodiments, the insulating tube 150 may be releasably coupled to the clip body 120 such as by friction fit.

The insulating tube 150 is shown in its compressed configuration disposed on a guidewire 50 along with the clip body 120 in FIG. 3A. The insulating tube 150 may be loaded onto the guidewire 50 when the insulating tube 150 is in its compressed configuration. When the first arm 121 and the second arm 122 of the clip body 120 are pinched into the open position, the clip body 120 and the insulating tube 150 may be placed laterally onto the guidewire with the insulating tube positioned proximal to the clip body 120. Transitioning the first and second arms 121,122 (only the first arm 121 is visible from the side) of the clip body 120 into the closed position consequently holds the insulating tube 150 in position. Once loaded onto the guidewire 50, the insulating tube 150 is positioned proximally from the clip body 120.

The insulating tube 150 has a loose fit over the guidewire to ensure at least partial circumferential coverage of the guidewire 50. The insulating tube 150 is dimensioned to provide clearance spacing over the outer diameter of the guidewire 50 sufficient to maintain electrical isolation. The inner diameter of the insulating tube 150 may range from 1.0 millimeters (0.03937 inches) to 30 millimeters (1.1811 inches).

FIG. 3B illustrates a bottom view of the insulating tube 150. In some embodiments, the insulating tube 150 may include a longitudinal split 155 along the entire length of the insulating tube 150, as shown in FIG. 3B. The longitudinal split 155 serves to facilitate loading the insulating tube 150 onto the guidewire by enabling lateral placement of the insulating tube 150 when the clip body 120 is in an open position. More specifically, the longitudinal split 155 enables the insulating tube 150 to be placed directly over the guidewire 50 at any location along the guidewire 50 without needing to be tracked axially from the proximal end of the guidewire 50, reducing interference with existing connections between the components of the pacing system.

The insulating tube 150 is shown in its extended configuration in FIG. 3C. Once the insulating tube 150 is placed onto the guidewire 50 and the clip body 120 is in the closed position, the insulating tube 150 may then be transitioned from its compressed configuration. To transition the insulating tube 150 from its compressed configuration to the expanded configuration, the insulating tube may be stretched proximally from its initial compressed configuration to cover the exposed portion of the guidewire 50. The accordion-like structure of the insulating tube 150 allows the insulating tube 150 to transition from its initial compressed configuration to the extended configuration while maintaining consistent circumferential coverage of the guidewire 50.

To secure a positive terminal connection necessary for establishing the electrical circuit of a pacing system, a user may position the pacing clip device 105 onto the guidewire. The user may begin by transitioning the clip body 120 to the open position. More specifically, the user may pinch the first and second arms 121, 122 to move them apart relative to each other along the central axis of the guidewire 50 (e.g., the user “pinches” the top portion 123 of first arm 121 and the top portion 124 of the second arm 122 closer together). When the user pinches the top portions 123, 124 of the first and second arms 121, 122 together, the first bottom portion 125 of the first arm 121 and the second bottom portion 126 move apart relative to each other and away from the central axis of the guidewire 50.

After the clip body 120 has been transitioned into the open position, the user may then transition the clip body 120 into the closed position over the guidewire 50. To transition the clip body 120 into the closed position, the user may pivot the first arm 121 and the second arm 122 relatively toward each other. In other words, the user releases the “pinch”. The spring mechanism 180 then maintains the closed position.

By transitioning the clip body 120 into the closed position, at least one semicircular cutout 130 on the conductive connector 115 may be electrically engaged with the core wire 144 between the turns of the guidewire coil 140. In some embodiments, the guidewire coil 140 is sufficiently compressible, such that the spaces 142 between the turns of the guidewire coil 140 at the location where the prongs 130a, 130b of the conductive connector 115 are engaged with the turns are widened, while the remaining turns of the guidewire coil 140 are compressed once the clip body 120 has been transitioned into its closed position. In the closed position, the prongs 130a, 130b are in contact with the core wire 144.

Once the clip body 120 is in the closed position, the insulating tube 150, attached to the clip body 120 and disposed laterally over the guidewire 50 in its compressed configuration, may be extended longitudinally along the guidewire 50 in a proximal direction.

Once the insulating tube 150 has been proximally and longitudinally extended into its extended configuration, the user may then connect the guidewire 50 to the positive terminal of the EPG. Either before or after the guidewire 50 has been connected to the positive terminal of the EPG, the other components of a conventional pacing system may be connected, in a conventional manner, to create the needed electrical circuit and ready the pacing system to deliver electrical stimuli.

It should be understood that this disclosure is, in many respects, only illustrative. Changes may be made in details, particularly in matters of shape and size, without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A pacing clip device for use with a guidewire during transcatheter procedures, comprising:
a clip body having a first arm pivotally connected to a second arm to transition between an open position and a closed position;
a conductive connector housed within at least one of the first and second arms and having at least one semicircular cutout configured to electrically engage the guidewire;
a spring mechanism configured to hold the first and second arms in the closed position; and
an insulating tube extending proximally from the clip body.

2. The pacing clip device of claim 1, wherein the semicircular cutout is configured to establish electrical contact with a core wire of the guidewire between insulating wrappings of the guidewire when the clip body is in the closed position.

3. The pacing clip of claim 2, wherein the conductive connector includes a distal prong and a proximal prong individually configured to engage a space between turns of the insulating wrappings on the guidewire and mechanically and electrically contact the core wire.

4. The pacing clip device of claim 1, wherein:
the first arm comprises a first top portion and a first bottom portion, and the second arm comprises a second top portion and a second bottom portion;
wherein the first top portion and the second top portion are moved toward a central axis of the guidewire and the first bottom portion and the second bottom portion are moved apart relative to the central axis of the guidewire when the clip body is in the open position; and
wherein the first top portion and the second top portion are moved apart relative to the central axis of the guidewire and the first bottom portion and the second bottom portion are moved toward the central axis of the guidewire when the clip body is in the closed position.

5. The pacing clip device of claim 1, wherein the insulating tube comprises an accordion-like structure with alternating ridges and valleys configured to transition from a compressed configuration to an extended configuration.

6. The pacing clip device of claim 5, wherein the insulating tube is in the compressed configuration when the pacing clip device is loaded onto the guidewire.

7. The pacing clip device of claim 5, wherein the insulating tube is configured to cover at least an exposed portion of a core wire of the guidewire when the insulating tube is in the extended configuration.

8. The pacing clip device of claim 1, wherein the insulating tube comprises:
a proximal portion having an accordion-like structure with alternating ridges and valleys configured to extend proximally along the guidewire; and
a distal portion lacking the accordion-like structure configured to be coupled with the clip body.

9. The pacing clip device of claim 1, wherein the insulating tube includes a longitudinal split extending along its length to allow for lateral placement of the insulating tube onto the guidewire when the clip body is in the open position.

10. A method of attaching a pacing clip device to a pacing system during a transcatheter procedure, comprising:
attaching a clip body to a guidewire, the clip body including:
a first pivotable arm and a second pivotable arm;
a conductive connector disposed within at least one of the first and second pivotable arms, the conductive connector having at least one end surface defining a cutout;
transitioning the clip body to an open position;
positioning the clip body onto the guidewire while in the open position;
transitioning the first and second pivotable arms to a closed position by pivoting the first pivotable arm and the second pivotable arm relative to each other;
wherein at least one cutout is engaged with a core wire of the guidewire, when the clip body is in the closed position; and
extending an insulating tube proximally from the clip body along the guidewire from a compressed configuration to an extended configuration over an exposed proximal portion of the guidewire.

11. The method of claim 10, wherein the conductive connector includes a distal prong and a proximal prong each defining the at least one end surface defining the cutout, wherein the distal and proximal prongs are configured to be inserted between insulating wrappings of the guidewire when the clip body is in the closed position.

12. The method of claim 11, wherein the distal prong and the proximal prong are in contact with the core wire of the guidewire when the clip body is in the closed position.

13. The method of claim 10, wherein extending the insulating tube comprises transitioning the insulating tube from the compressed configuration to the extended configuration to cover at least an exposed portion of the core wire of the guidewire.

14. The method of claim 10, wherein positioning the clip body further comprises laterally placing the clip body onto the guidewire and laterally placing the insulating tube onto the guidewire through a longitudinal split in the insulating tube.

15. A system to establish an electrical connection in a pacing system, comprising:
a guidewire having a guidewire coil wrapped around a core wire; and
a pacing clip device comprising:
a clip body having a first arm and a second arm pivotally connected to transition between an open position and a closed position;
a conductive connector housed within at least one of the first and second arms and having at least one end with a semicircular cutout configured to engage the core wire between turns of the guidewire coil;
a spring mechanism configured to hold the first and second arms in the closed position; and
an insulating tube extending proximally from the clip body.

16. The system of claim 15, wherein the semicircular cutouts are configured to mechanically and electrically engage with the core wire between wrappings of the guidewire coil of the guidewire when the clip body is in the closed position.

17. The system of claim 15, wherein the insulating tube includes a longitudinal split configured to enable lateral placement of the insulating tube onto the guidewire.

18. The system of claim 15, the insulating tube comprises an accordion-like structure with alternating ridges and valleys configured to extend proximally along the guidewire after transitioning the insulating tube from a compressed configuration to an extended configuration.

19. The system of claim 18, wherein the insulating tube is configured to cover an exposed portion of the core wire of the guidewire when the insulating tube is in the extended configuration.

20. The system of claim 15, wherein the first and second arms are configured to transition between the open position for lateral placement of the guidewire and the closed position wherein the conductive connector engages the guidewire to establish electrical contact.
